(19) United States
(12) Patent Application Publication
(10) Pub. No.: US 2026/0076793 A1
(43) Pub. Date: Mar. 19, 2026

(54) Combination of Variable Power Lenses for Accommodating Intraocular Lens

(71) Applicant: AKKOLENS INTERNATIONAL B.V., Breda (NL)

(72) Inventors: Michiel Christiaan Rombach, Breda (NL); Willem Pieter Van Lawick, Breda (NL)

(21) Appl. No.: 19/110,603

(22) PCT Filed: Sep. 12, 2023
(86) PCT No.: PCT/NL2023/050467

§ 371 Date: Mar. 11, 2025

(30) Foreign Application Priority Data
Sep. 12, 2022 (NL) 2033002

(51) Int. Cl. A61F 2/16; A61F 2/48

(52) U.S. Cl. CPC A61F 2/1651; A61F 2/482; A61F 2002/1682; A61F 2002/1689; A61F 2250/0018

(57) ABSTRACT

Accommodating intraocular lenses with a combination of variable power lenses including, firstly, a variable lens with cubic surfaces fitted onto two optical elements to vary focal power by lateral shift of the elements and, secondly, a variable lens with spherical lenses fitted onto the same elements to vary focal power by axial shift of the elements. A mechanical construction with hinges provides both the shifts of the elements.

### CROSS-REFERENCE TO RELATED APPLICATIONS

This application is the United States national phase of International Application No. PCT/NL2023/050467, filed Sep. 12, 2023, and claims priority to The Netherlands Patent Application No. 2033002, filed Sep. 12, 2022, the disclosures of which are hereby incorporated by reference in their entireties.

### BACKGROUND OF THE INVENTION

### Field of the Invention

Accommodating intraocular lenses provide the eye with variable focus power, also: ‘variable power’, by a variable power lens, ‘variable lens’, to restore accommodation of the eye and, generally, but not necessarily so, also correct refraction of the eye from which the natural lens is removed.

### Description of Related Art

Accommodating intraocular lens constructions can comprise multiple optical elements each fitted with at least one cubic, rotationally asymmetrical, free-form, optical surface. The combination of two, such cubic optical surfaces provide a lens of variable power of which the degree of power depends on the degree of mutual shift of the optical elements in an opposite lateral direction, meaning: an opposite shift in a direction perpendicular to the optical axis. Such intraocular variable lenses are disclosed in, for example, US2008046076 and WO2005084587. Other accommodating intraocular lens constructions comprise optical elements each fitted with largely spherical optical surface. The combination of, for example, a positive spherical surface and a negative spherical surface provides a lens of variable power with the degree of power depending on the degree of mutual opposite axial shifts of the elements along the optical axis with the element with a positive spherical surface shifting in the anterior direction, the direction towards the cornea and the element with a negative spherical surface shifting in the posterior direction, the direction towards the retina as disclosed in, for example, WO2011062486, US20020002404. Alternatively, both elements can be fitted with a positive spherical surface and both elements shift in the anterior direction which shift provides variable power of which the degree of power depends on the degree of axial shifts.

Note that accommodating intraocular lenses can also comprise fluid-filled variable lenses as in, for example, US1562252260 and US2019374333. Such fluid-filled variable optics can also be included into the constructions disclosed in the present document.

### SUMMARY OF THE INVENTION

It is a first object of the present invention to provide an improved lens construction.

It is a second object of the present invention to provide an intraocular lens construction with enhanced optical accommodation.

This present invention discloses thereto an accommodating intraocular lens construction, comprising an optical axis, with the lens construction comprising
at least two optical elements which elements are mutually coupled by at least one elastic haptic and
wherein at least one optical element, preferably each optical element, comprises
   at least one optical surface comprising an at least partly cubic function shaped or free-formed shape; and
   at least one optical surface comprising an at least partly spherical shape;
wherein the at least one haptic is configured for co-action with at least one driving means in an eye, wherein the driving means comprise at least one natural driving means and/or artificial driving means;
and wherein the at least one haptic comprises at least one hinge configured to, upon a shift of said driving means, translate shift of said driving means in the eye into lateral shift and/or axial shift of the at least two optical elements (with respect to each other), wherein lateral shift is a shift in a direction perpendicular to the optical axis and wherein axial shift of the optical elements is a shift in a direction along the optical axis.

The lateral shift and/or axial shift can be understood as movements in a lateral and/or axial direction. The lateral direction is to be understood as a direction perpendicular to the optical axis. The axial direction is to be understood as a direction along the optical axis.

The lens construction comprises, firstly, a combination of at least two partly cubic optically shaped surfaces, preferably at least two substantially cubic optical shaped surfaces. The at least two partly cubic optical shaped surfaces provide a lens of variable optical defocus power of which the degree of optical defocus power depends on the degree of mutually opposite shifts of the elements in a lateral direction.

The lens construction, secondly, also comprises a combination of at least two at least partly spherical optical shaped surfaces, preferably at least two substantially spherical optical shaped surfaces. The at least two partly spheric optical shaped surfaces provide a lens of variable optical power of which the degree of power depends on the degree of shifts of the elements in an axial direction which shifts can be in the same direction along the axis or, alternatively, in mutually opposite directions along the axis.

The haptic can be configured to translate a shift of the driving means into any combination of shift of the elements in a lateral direction and shift of the elements in an axial direction.

In an embodiment, a shift of at least one driving means may be a lateral and/or an axial shift.

The present invention relates to an accommodating intraocular lens construction with combinations of cubic variable lenses and spherical variable lenses fitted onto two optical elements (to vary focal power). The combination includes at least one cubic variable lens of which at least one cubic optical surface is fitted onto at least one element and preferably onto each element. The combination also includes at least one spherical variable lens of which at least one spherical lens is fitted onto at least one element and preferably onto each element.

The cubic variable lens varies power by mutual shift of the cubic surface in an opposite lateral direction, a direction perpendicular to the optical axis. The spherical variable lens varies power by a shift of at least one spherical surface in an axial direction, a direction along the optical axis.

Where in this document reference is made to cubic surface and/or cubic variable lenses, this can be interpreted as comprising a surface formed according to the shape of a graph resulting from a cubic formula. This can be referred to as cubic, cubic optical shape in this document and/or as (partially) cubic function shaped and/or free-formed and/or cubic optical shaped.

The lens construction Includes a mechanical construction into which the elements comprising the variable lenses can be fitted. The mechanical construction comprises at least one elastic haptic for positioning the lens construction in the eye and to transfer shift of a driving means in the eye to at least one element, preferably each element. The haptic may comprise a combination of multiple hinges, in particular two hinges with the hinges providing translation of a lateral shift of the driving means into a lateral shift of at least one element and translation of the same lateral shift of the driving means into an axial shift of at least one element. Or, in an embodiment, at least one haptic may comprise a single hinge which provides for two functions of shifting the optical elements laterally and axially.

So, in an embodiment, the present invention relates to an accommodating intraocular lens construction, having an optical axis, with the lens constructions comprising at least two optical elements which elements can be mutually coupled by at least one elastic haptic with each element fitted with at least one optical surface according to a cubic optical shape and with each element fitted with at least one optical surface according to a spherical optical shape wherein the haptic comprises at least one hinge adapted to translate shift of driving means in the eye into a combination of lateral shift, meaning: a shift perpendicular to the optical axis, and axial shift, meaning: a shift along the optical axis, of the elements.

Both optical elements can comprise a spherical optical surface of a positive optical power. In combination with a mechanical construction, such as a hinge and/or haptic, the intraocular lens construction according to the present invention can be configured to shift both elements in the same direction along the optical axis. Alternatively, one optical element, generally the anterior optical element, can spherical optical surface of a positive optical power with the other element comprising a spherical optical surface of a negative optical power with the haptic of the mechanical construction adapted to shift both elements in opposite directions along the optical axis. A negative spherical surface can also provide correction for the said additional power of the overpowered positive spherical surface on the anterior optical element to secure emmetropia to the eye when the lens construction is at the resting state, meaning: provide sharp vision to the eye at far at the resting state of the lens construction. Note that the cubic and spherical optical surfaces can be differently distributed over the four surfaces of the optical elements or, alternatively, optical surfaces can be combined. For example a spherical surface can be combined with a free-form cubic surface.

The combination of at least two largely cubic optically shaped surfaces, if applied, provides a lens of variable optical defocus power of which the degree of optical defocus power depends on the degree of mutually opposite shifts of the elements in a lateral direction. The combination of at least two largely spherical optically shaped surfaces, if applied, provides a lens of variable optical power of which the degree of optical power depends on the degree of shifts of the elements in an axial direction. Both elements can comprise a positive spherical surface with a mechanical construction, haptic, configured to shift both elements in the same direction along the optical axis, or, alternatively, one optical element comprises a positive spherical surface with the other element comprising a negative spherical surface with mechanical construction adapted to shift both elements in opposite directions along the optical axis.

At least one haptic can translate a shift of the at least one driving means into lateral shifts of the optical elements in mutually opposite directions and the haptic can translate a shift of the driving means into axial shifts of the optical elements in mutually opposite directions. Alternatively, the haptic may translate a shift of at least one driving means into axial shift of the optical elements in the same direction.

At least one haptic can comprise a combination of two independent hinges to, independently, provide the lateral and the axial shift of the optical elements, or, alternatively, the elastic haptic can comprise a single hinge adapted to provide both the lateral and axial shift of the optical elements. Note that driving means in the eye, for example the ciliary muscle, can shift in a lateral and axial direction. At least one haptic can be configured such that the shift in lateral direction can be translated into a shift of the elements in a lateral and/or axial direction. At least one haptic can be configured such that also the shift in axial direction can be translated into a shift of the elements in a lateral direction.

The driving means of the lens construction can be a natural component of the eye, for example the ciliary muscle and/or the capsular bag, or any other natural driving component in the eye or any combination of such natural driving means, or, alternatively, the driving means can be an artificial driving means, for example an electro-mechanical MEMS component.

The lens construction can be designed, by addition of spherical optical power, to restore a combination of accommodation and refraction of the human eye. Also, each optical element can comprises at least one additional free-form surface to provide correction of or enhancement of any variable aberration (other than focus), for example, variable aspheric aberrations and/or variable toric aberrations.

The lens construction can fully replace the functions of the natural lens after removal of the natural lens. In an embodiment, the lens construction according to the present invention is an add-on lens. Such lens can restore, as an add-on lens, accommodation in combination with the natural lens or in combination with a fixed focal lens which replaces the natural lens with, for example, a standard monofocal intraocular lens positioned in the capsular bag with the lens construction positioned at the sulcal plane, in front of the capsular bag.

The at least one hinge may be elastic. The elasticity ensures that the hinge can be temporarily deformed by compression or elongation, while retaining the ability to return to its original shape prior to deformation. As such, the hinge can be for example compressed by a ciliary muscle and/or a capsular bag resulting in a shift of the optical elements with respect to each other.

The at least one hinge may comprise an anterior hinge and a posterior hinge, wherein the anterior hinge is attached on a first side to an anterior optical element of the at least two optical elements, and wherein the posterior hinge may be attached on a second side to a posterior optical element of the at least two optical elements, and wherein the first side of the anterior optical element and second side of the posterior optical element are located on opposite outer edges of the lens construction.

In line with the above, an anterior stiff transfer component may be attached on a second side to the anterior optical element, and a posterior stiff transfer component may be attached on a first side to the posterior optical element, and wherein the second side of the anterior optical element and the first side of the posterior optical element are located on opposite outer edges of the lens construction. Advantageously, this allows the shift of the anterior optical element in a direction perpendicular to the optical axis, while the posterior optical element is shifted in an opposite direction. Based on the compressive force enacted by the driving means, this shift is adjustable.

Stiff herein means not elastic. The stiff transfer components are at least less elastic as the hinges.

Preferably, the at least one optical element comprises an anterior optical element and a posterior optical element, and a posterior optical surface of the anterior optical element and an anterior optical surface of the posterior optical element are at least partly cubic function shaped.

### BRIEF DESCRIPTION OF THE DRAWINGS

The terms Fig., Figs., Figure, and Figures are used interchangeably in the specification to refer to the corresponding figures in the drawings.

FIG. 1 is a top/cut view showing an accommodating intraocular lens construction;

FIG. 2 shows a side/cut view of the lens construction as of FIG. 1;

FIG. 3 shows a side/cut view of the first embodiment of the lens construction with the construction in a compressed state; and

FIG. 4 shows a side/cut view of the second embodiment of the lens construction in a compressed state.

### DETAILED DESCRIPTION OF THE INVENTION

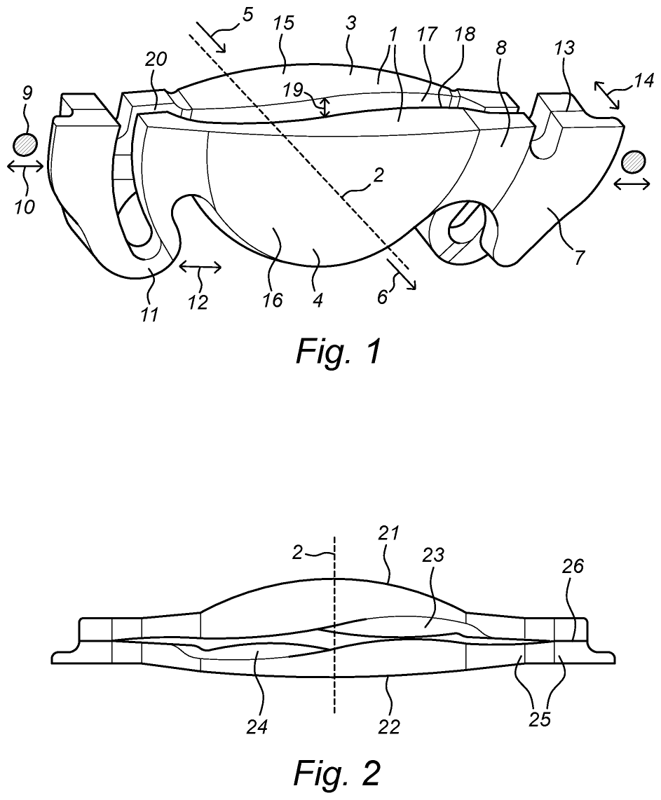

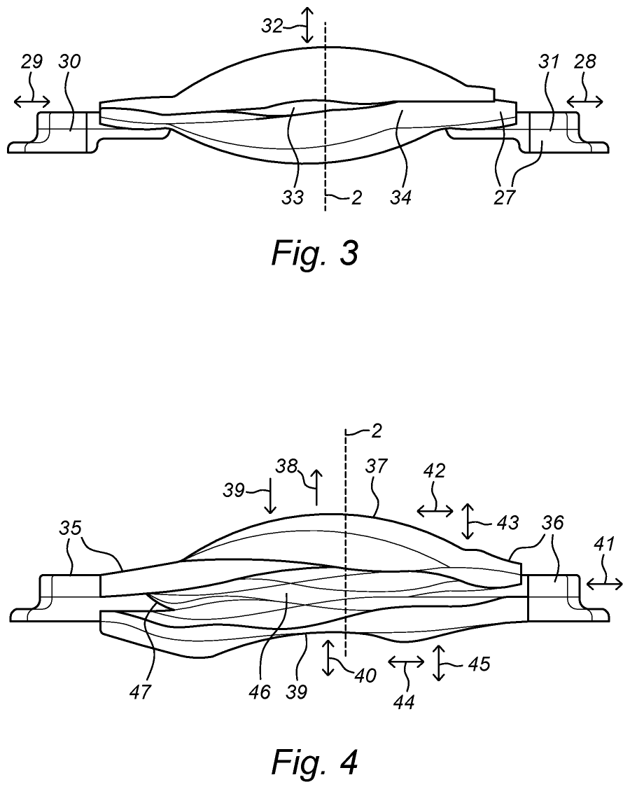

FIG. 1, top/cut view (note that the lens construction is, largely, symmetrical so reference numbers are transferable to opposite sections of the illustrations) shows an accommodating intraocular lens construction comprising a variable power optical lens with fixed power optical surfaces, 1, having an optical axis, 2, which lens comprises an anterior element, 3, (facing the cornea of the eye) and a posterior element, 4, (facing the retina of the eye). The arrow, 5, indicates the direction of the incoming light from the cornea and the arrow, 6, the direction of the outgoing light towards the retina. The mechanical construction includes elastic haptics, 7, to position the construction in the eye and to transfer lateral shift of driving means via elastically stiff transfer components, 8, to the optical elements, with the shift, 10, generated by natural driving means in the eye, 9. The elastic haptics include a first set of elastical hinges, 11, to provide for lateral shift, 12, of the optical elements. The haptics also include a second set of hinges, 13, to provide for axial shift, 14, of the optical elements. The elements each comprise at least one rotational symmetrical spherical optical surface, 15,16. These can be both positive spherical surfaces (as in the first embodiment, FIG. 3), or, alternatively, can be a positive and a negative spherical lens (as in the second embodiment, FIG. 4). Each of the elements also comprise at least one, rotationally asymmetrical, free-form, largely cubic shaped optical surface, 17,18, which cubic surfaces form the lining of, in this example, the medial intra-lenticular space, 19, and, at least partly, the lateral intra-lenticular spaces, 20.

FIG. 2, shows a side/cut view of the lens construction as in FIG. 1. Note the spherical surfaces, 21,22, the cubic surfaces, 23,24, the first set of hinges, 25, providing lateral shifts of the elements and the second set of hinges, 26, providing axial shift of the elements.

FIG. 3 shows a side/cut view of the first embodiment of the lens construction with two positive spherical optical surfaces with the construction in a compressed state. The first set of hinges, 27, provide lateral shift of the optical elements in mutually opposite directions, 28,29. Also, the second set of hinges, 30,31, provide shift of at least one optical element in an axial direction, 32. Axial shifts can narrow the intra-lenticular space, 33, and narrow the lateral intra-lenticular spaces, 34.

FIG. 4 shows a side/cut view of the second embodiment of the lens construction, in a compressed state, in which the first set of hinges, 35, 36, provide lateral shift of the elements (similar to the embodiment in FIG. 3). However, in this second embodiment the second set of hinges provide for widening of the intra-lenticular space. The anterior optical element comprises an optically overpowered spherical lens, 37, meaning: a lens with an additional positive power on top of the refractive power required for the particular eye. The overpowered lens shifts forward, 38, along the optical axis at lateral compression of the lens construction by the driving means providing accommodating to the eye and backward, 39, during relaxation of the driving means providing des-accommodation to the eye. The posterior optical element comprises a negative spherical lens, 39, which shifts, 40, along the optical axis which backward shift also provides accommodation to the eye. Arrows indicate shifts of driving means, 41, lateral shifts of the anterior element, 42, axial shifts of the anterior optical element, 43, lateral shifts of the posterior element, 44, and axial shifts of the posterior optical element, 45. Note that in this embodiment, during lateral compression, the medial intra-lenticular space, 46, and the lateral intraocular spaces, 47, widen, with the degree of widening depending on the degree of lateral compression of the lens construction.

It will be clear that the invention is not limited to the exemplary embodiments which are illustrated and described here, but that countless variants are possible within the framework of the attached claims, which will be obvious to the person skilled in the art. In this case, it is conceivable for different inventive concepts and/or technical measures of the above-described variant embodiments to be completely or partly combined without departing from the inventive idea described in the attached claims.

The verb ‘comprise’ and its conjugations as used in this patent document are understood to mean not only ‘comprise’, but to also include the expressions ‘contain’, ‘substantially contain’, ‘formed by’ and conjugations thereof.

1. An accommodating intraocular lens construction, comprising an optical axis, with the lens construction comprising
at least two optical elements which elements are mutually coupled by at least one elastic haptic and
wherein at least one optical element, preferably each optical element, comprises
at least one optical surface comprising an at least partly cubic function shaped or free-formed shape; and
at least one optical surface comprising an at least partly spherical shape;
wherein the at least one haptic is configured for co-action with at least one driving means in an eye, wherein the driving means comprise at least one natural driving means and/or artificial driving means;
and wherein the at least one haptic comprises at least one hinge configured to, upon a shift of said driving means, translate shift of said driving means in the eye into lateral shift and/or axial shift of the at least two optical elements, wherein lateral shift is a shift in a direction perpendicular to the optical axis and wherein axial shift of the optical elements is a shift in a direction along the optical axis.

2. The lens construction according to claim 1, wherein at least one haptic is configured to translate a shift of the driving means into lateral shifts of the optical elements in mutually opposite directions.

3. The lens construction according to claim 1, wherein at least one haptic is configured to translate a shift of the driving means into axial shifts of the optical elements in mutually opposite directions.

4. The lens construction according to claim 1, wherein at least one haptic is configured to translate a shift of the driving means into axial shifts of the optical elements in the same direction.

5. The lens construction according to claim 1, wherein at least one haptic is configured to translate a shift of the driving means into a combination of shift of the elements in a lateral direction and shift of the elements in an axial direction.

6. The lens construction according to claim 1, wherein the driving means comprises a natural driving means chosen from the group of: ciliary muscle and/or a capsular bag in the eye or any combination of such natural driving means.

7. The lens construction according to claim 1, wherein the driving means comprises an artificial driving means comprising at least one electro-mechanical MEMS component.

8. The lens construction according to claim 1, wherein at least one haptic comprises two hinges configured to independently provide the lateral and the axial shift of the optical elements.

9. The lens construction according to claim 1, wherein at least one haptic comprises a single hinge, wherein the single is configured to provide both the lateral and axial shift of the optical elements.

10. The lens construction according to claim 1, wherein at least one haptic can be configured such that also a shift in axial direction of at least one driving means is translated into a shift of the elements in a lateral direction.

11. The lens construction according to claim 1, wherein at least one, preferably each, optical element comprises at least one additional free-form surface configured to provide correction of or enhancement of any variable aberration, such as variable aspheric aberrations and/or variable toric aberrations.

12. The lens construction according to claim 1, wherein the lens construction is configured to at least partly restore the accommodation of the human eye.

13. The lens construction according to claim 1, wherein the lens construction is configured to at least partly restore a combination of accommodation and refraction of the human eye.

14. The lens construction according to claim 1, wherein the at least one hinge is elastic.

15. The lens construction according to a claim 1, wherein the at least one hinge comprises an anterior hinge and a posterior hinge,
wherein the anterior hinge is attached on a first side to an anterior optical element of the at least two optical elements, and wherein the posterior hinge is attached on a second side to a posterior optical element of the at least two optical elements, and
wherein the first side of the anterior optical element and second side of the posterior optical element are located on opposite outer edges of the lens construction.

16. The lens construction according to claim 15, wherein an anterior stiff transfer component is attached on a second side to the anterior optical element, and
wherein a posterior stiff transfer component is attached on a first side to the posterior optical element, and
wherein the second side of the anterior optical element and the first side of the posterior optical element are located on opposite outer edges of the lens construction.

17. The lens construction according to claim 1, wherein the at least one optical element comprises an anterior optical element and a posterior optical element, and
wherein a posterior optical surface of the anterior optical element and an anterior optical surface of the posterior optical element are at least partly cubic function shaped.
